(19) United States
(12) Patent Application Publication
(10) Pub. No.: US 2022/0071249 A1
(43) Pub. Date: Mar. 10, 2022

(54) EXTRACTED AND FERMENTED COMPOSITION OF COFFEE CHERRY PULP AND SKIN AND METHOD FOR PRODUCING SAME

(71) Applicant: KIRIN HOLDINGS KABUSHIKI KAISHA, Tokyo (JP)

(72) Inventors: Sayaka TSUJI, Tokyo-to (JP); Hiroki FUJIWARA, Tokyo-to (JP); Keiko NAKASHIMA, Tokyo-to (JP)

(73) Assignee: KIRIN HOLDINGS KABUSHIKI KAISHA, Tokyo (JP)

(21) Appl. No.: 17/418,658

(22) PCT Filed: Dec. 27, 2019
(86) PCT No.: PCT/JP2019/051500

§ 371 Date: Jun. 25, 2021

(30) Foreign Application Priority Data
Dec. 28, 2018 (JP) 2018-247991
Feb. 26, 2019 (JP) 2019-033224

(51) Int. Cl. A23L 19/00; A23L 5/20; A23L 2/02; A23L 2/38

(52) U.S. Cl. CPC A23L 19/09; A23L 2/382; A23L 2/02; A23L 5/28

(57) ABSTRACT

The present invention provides an extracted and fermented composition of coffee cherry pulp and skin having an increased β-damascenone content and a method for producing the same. More specifically, in the method for producing an extracted and fermented composition of coffee cherry pulp and skin, a fermentation step using lactic acid bacteria is included.

### CROSS REFERENCE TO RELATED APPLICATIONS

The present patent application claims priority based on Japanese Patent Application No. 2018-247991 filed on Dec. 28, 2018 and Japanese Patent Application No, 2019-033224 filed on Feb. 26, 2019, and the whole disclosures of the earlier patent applications are hereby incorporated by reference as a part of this description.

### TECHNICAL FIELD

The present invention relates to an extracted and fermented composition of coffee cherry pulp and skin and a method for producing the same, and more particularly to an extracted and fermented composition of coffee cherry pulp and skin having an increased β-damascenone content and a method for producing the same.

### BACKGROUND ART

A coffee plant is a broad-leaved shrub belonging to the genus Coffea in the family Rubiaceae. Coffee fruits are oval fruits that coffee plants bear and are green at the beginning and magenta when ripe. Some varieties rarely bear yellow fruits. Coffee fruits are also called coffee cherries. The coffee cherry contains two seeds facing each other, and the part of the two seeds is called coffee beans. In addition, a coffee cherry has a structure of outer skin, pulp, endocarp (parchment), silver skin, and seeds (coffee beans) from the outside.

Among them, only coffee beans are used as a raw material for producing coffee beverages. Therefore, a lot of pulp and skin are by-produced in the refining process for obtaining coffee beans from coffee cherries. At present, there is almost no use for the by-products, which are disposed, and a new use is expected for them.

On the other hand, PTL 1 describes green coffee beans having a β-damascenone content of 5 ppb.

However, no report has been made on an extracted and fermented composition of coffee cherry pulp and skin which contains a high content of β-damascenone.

### PRIOR ART DOCUMENT

### Patent Document

[Patent Document 1] JP 2018-057369 A

### SUMMARY OF THE INVENTION

As a result of a study on the extracted and fermented composition of coffee cherry pulp and skin, the present inventors have found that the composition contains β-damascenone in a high content as an ingredient thereof. Furthermore, they have found that the content of β-damascenone is increased by including a fermentation step using lactic acid bacteria in a method for producing an extracted and fermented composition of coffee cherry pulp and skin. The present invention is based on these findings.

Thus, an object of the present invention is to provide an extracted and fermented composition of coffee cherry pulp and skin containing a high content of β-damascenone and a method for producing the same.

According to the present invention, the following inventions are provided.

(1) An extracted and fermented composition of coffee cherry pulp and skin, comprising β-damascenone at 9.6 ppb or more.
(2) The extracted and fermented composition of coffee cherry pulp and skin according to (1), wherein the composition contains 50 times or more of β-damascenone as compared to an unfermented coffee cherry pulp and skin extract liquid.
(3) A method for producing an extracted and fermented composition of coffee cherry pulp and skin, comprising subjecting pulp and skin of coffee cherry to an extraction process and a fermentation process using lactic acid bacteria.
(4) The production method according to (3), wherein the method comprises a step of fermenting a coffee cherry pulp and skin extract liquid using lactic acid bacteria.
(5) The production method according to (3) or (4), wherein the lactic acid bacteria are at least one selected from the group consisting of the genus Lactobacillus, the genus Oenococcus, the genus Pediococcus, and the genus Lactococcus.
(6) The production method according to any one of (3) to (5), further comprising a yeast fermentation process.
(7) The production method according to any one of (3) to (6), wherein the method comprises a step of fermenting using yeast after the step of fermenting a coffee cherry pulp and skin extract liquid using lactic acid bacteria.
(8) The production method according to any one of (3) to (7), further comprising a treatment with a filter aid.
(9) The production method according to any one of (3) to (8), further comprising a step of treating the coffee cherry pulp and skin extract liquid with a filter aid before the step of fermenting the coffee cherry pulp and skin extract liquid using lactic acid bacteria.
(10) The production method according to any one of (3) to (9), further comprising a concentration process.
(11) The method for producing the composition according to any one of (3) to (10), further comprising a step of concentrating a coffee cherry pulp and skin extract liquid before the step of fermenting the coffee cherry pulp and skin extract liquid using lactic acid bacteria.
(12) An extracted and fermented composition of coffee cherry pulp and skin obtained by the production method according to any one of (3) to (11).
(13) A beverage comprising the extracted and fermented composition of coffee cherry pulp and skin according to any one of (1), (2) and (12).
(14) The beverage according to (13), wherein the beverage is a packaged beverage.
(15) A method for increasing a β-damascenone content in an extracted and fermented composition of coffee cherry pulp and skin, comprising subjecting coffee cherry pulp and skin to an extraction process and a fermentation process using lactic acid bacteria.
(16) The method according to (15), wherein the method comprises a step of fermenting a coffee cherry pulp and skin extract liquid using lactic acid bacteria.

According to the present invention, the S-damascenone content can be increased, as described above, by including a fermentation step using lactic acid bacteria in the method for producing an extracted and fermented composition of coffee cherry pulp and skin.

### DETAILED DESCRIPTION OF THE INVENTION

One of the features of the extracted and fermented composition of coffee cherry pulp and skin of the present invention is a high content of 1-damascenone. Here, the “extracted and fermented composition of coffee cherry pulp and skin” refers to a composition obtained by fermenting an extract liquid of pulp and skin of coffee cherry or an extract liquid of fermented pulp and skin of coffee cherry (Hereinafter, also referred to as “extracted and fermented composition” or “composition of the present invention”). In the present invention, the content of S-damascenone in the composition can be increased by including the step of fermenting the coffee cherry pulp and skin extract liquid or the coffee cherry pulp and skin using lactic acid bacteria in the method for producing the composition of the present invention,

### Extracted and Fermented Composition of Coffee Cherry Pulp and Skin

The extracted and fermented composition of coffee cherry pulp and skin of the present invention contains β-damascenone in a high content in the composition. β-Damascenone is also described as (E)-1-(2,6,6-trimethyl-1,3-cyclohexadien-1-yl)-2-buten-1-one, and is one of the aroma components of coffee, which has a sweet fragrance like honey. The β-damascenone content in the composition of the present invention is, for example, 9.6 ppb or more, preferably 12 ppb or more, and more preferably 14.4 ppb or more. Here, “ppb” indicates parts per billion, and 1 ppb corresponds to 1 μg/L. The preferred lower limit of the β-damascenone content in the composition of the present invention is 9.6 ppb, preferably 12 ppb, more preferably 14.4 ppb, and the preferred upper limit is 120 ppb, and more preferably 60 ppb. The β-damascenone content in the composition is measured by gas chromatography-mass spectrometry (GC-MS). Such a measurement can be easily performed by using a commercially available device and a column (for example, BP-20 (manufactured by SGE Analytical Science Pty. Ltd)). The measurement can be performed, for example, under the following conditions. Equipment: 78908 (manufactured by Agilent Technologies, Inc.), JMS-Q1500 (manufactured by JEOL Ltd.). Column: BP-20 (manufactured by SGE Analytical Science Pty. Ltd), C measurement conditions: Oven temperature (initial: 60° C., hold 3 minutes, temperature rise: 3° C./min, 170° C., hold 3 minutes, 10° C./min, 245° C., hold 10 minutes), injection port setting (injection amount: 2 μL, mode: splitless 10: 1, temperature: 250° C., pressure: 162.35 kPa), total flow rate: 1 mL/min, septum purge flow rate: 3 mL/min, aux temperature: 240° C., MS measurement conditions (SIM/scan): SIM target ion: 190 (β-Damascenone), threshold: 10, MS zone temperature (Ion source temperature: 230° C., quadrupole temperature: 100° C.).

The extracted and fermented composition of coffee cherry pulp and skin of the present invention contains β-damascenone, for example, 50 times or more as compared to the unfermented coffee cherry pulp and skin extract liquid, preferably 80 times or more, and more preferably 100 times or more. The upper limit is, for example, 700 times, and preferably 400 times.

The pulp and skin of the present invention include outer skin and pulp, preferably consisting only of outer skin and pulp, and not containing seeds (coffee beans). The pulp and skin may be raw, frozen, or dried, and from an economical or industrial point of view, may be a large amount of raw pulp and skin by-produced by the following wet process during the separation of coffee beans,

### Method for Producing Extracted and Fermented Composition of Coffee Cherry Pulp and Skin

One of the features of the method for producing an extracted and fermented composition of coffee cherry pulp and skin of the present invention is subjecting pulp and skin of coffee cherry to an extraction process and a fermentation process using lactic acid bacteria. Hereinafter, the above-mentioned production method is described mainly on the followings: pulp and skin of coffee cherry are subjected to an extraction process, the coffee cherry pulp and skin extract liquid obtained is subjected to a fermentation process using lactic acid bacteria, and then, as desired, the fermented liquid using lactic acid bacteria of coffee cherry pulp and skin extract liquid is subjected to a yeast fermentation process. However, those skilled in the art can understand that the extracted and fermented composition of coffee cherry pulp and skin can be produced using each of the above processes even in a different order.

According to a preferred embodiment of the present invention, the production method of the present invention is a method for producing an extracted and fermented composition of coffee cherry pulp and skin, which comprises a step of fermenting the coffee cherry pulp and skin extract liquid using lactic acid bacteria. The method for producing the composition of the present invention is preferably a method for producing the composition containing β-damascenone at a high content.

According to a preferred embodiment of the present invention, the content of β-damascenone in the composition of the present invention can be increased by fermenting the coffee cherry pulp and skin extract liquid using lactic acid bacteria.

### <Step of Producing Coffee Cherry Pulp and Skin Extract Liquid>

The step of producing the coffee cherry pulp and skin extract liquid is not particularly limited, but includes a step of extracting the ingredients in the pulp and skin by mixing the pulp and skin, from which coffee beans have been separated and removed, with a solvent and allowing it to stand.

Further, according to another aspect of the present invention, step of producing the coffee cherry pulp and skin extract liquid is, for example, a step of juicing (for example, juicing by squeeze) the pulp and skin, from which coffee beans have been separated and removed, in the absence of a solvent. Thus, the coffee cherry pulp and skin extract liquid of the present invention also includes the juice of pulp and skin.

The method for separating coffee beans is not particularly limited, but usually includes a dry process and a wet process. In the dry process, after harvesting, the coffee cherries are first dried to a water content of 10 to 11% by weight. The coffee beans are then separated from the covering material (for example, outer skin, pulp, endocarp, and silver skin) using a hulling machine. The wet process, on the other hand, does not require drying of the coffee cherries. In the wet process method, coffee beans are mechanically separated from the outer skin and pulp, fermented, and then the layer of pulp material remaining on the beans is removed. After fermentation, the coffee beans are dried to a water content of about 12% by weight, hulled to separate the endocarp. By the above method, the pulp and skin of coffee cherry can be obtained.

The extraction time varies depending on the conditions of the pulp and skin, for example, degree of drying, size, extraction temperature, and the like, and is not particularly limited, but is for example, 5 minutes to 24 hours, preferably 15 minutes to 6 hours, and preferably 30 minutes to 2 hours. The extraction temperature varies depending on the conditions of the pulp and skin, for example, degree of drying, size, the extraction time, and the like, and is not particularly limited, but is, for example, 20 to 90° C., preferably 40 to 80° C., and preferably 50 to 70° C.

The solvent used for extraction is not particularly limited, but a solvent such as water, alcohols, ethers and esters, or a mixed solvent thereof can be used, and water is preferable.

The ratio of the pulp and skin to the solvent for extraction is not particularly limited, but from the viewpoint of practical convenience, raw material weight per 1 L of the solvent is, for example, 100 to 2000 g, preferably 300 to 1600 g, and further preferably 600 to 1300 g. The extraction concentration of 100 g/L or more enables preparation of a rich extract, which is advantageous in saving a large amount of energy and equipment during the subsequent concentration process. On the other hand, that of 2000 g/L or less is advantageous in simplifying the extraction operation and increasing the collected amount of the extract.

After completion of the extraction, it is preferable to separate the coffee cherry pulp and skin extract liquid from the pulp and skin. The separation method is not particularly limited, but can be performed by using a juicer, a centrifuge, or a filter, and it is preferable to squeeze with a juicer.

### <Step of Fermenting Coffee Cherry Pulp and Skin Extract Liquid or Coffee Cherry Pulp and Skin Using Lactic Acid Bacteria>

The method for producing the extracted and fermented composition of coffee cherry pulp and skin of the present invention preferably includes performing a fermentation process using lactic acid bacteria. Hereinafter, the step of fermenting the coffee cherry pulp and skin extract liquid using lactic acid bacteria will be described. However, the fermentation process using lactic acid bacteria may be performed by fermenting the pulp and skin of coffee cherry using lactic acid bacteria before the pulp and skin extraction process. Those skilled in the art can understand that the extracted and fermented composition of coffee cherry pulp and skin can be produced using each of the processes even in a different order.

According to a preferred embodiment of the present invention, the step of fermenting the coffee cherry pulp and skin extract liquid using lactic acid bacteria is not particularly limited, but includes a method for fermenting using lactic acid bacteria by mixing the coffee cherry pulp and skin extract liquid and the lactic acid bacteria and culturing lactic acid bacteria in the extract liquid.

The step of fermenting the coffee cherry pulp and skin extract liquid using lactic acid bacteria may be performed by directly fermenting the coffee cherry pulp and skin extract liquid obtained above, or may be performed after the step of treating the coffee cherry pulp and skin extract liquid with a filter aid and/or the step of concentrating the coffee cherry pulp and skin extract liquid.

The lactic acid bacteria used for fermentation are not particularly limited as long as it does not disturb the effects of the present invention, and examples thereof include Lactobacillus, Oenococcus, Pediococcus, Lactococcus and the like, or a combination thereof, and preferably Lactobacillus, Oenococcus, Lactococcus or a combination thereof. Examples of the lactic acid bacteria belonging to the genus Lactobacillus includes, for example, Lactobacillus plantarum, Lactobacillus rhamnosus, Lactobacillus paracasel, Lactobacillus amylovorus, Lactobacillus gasseri, Lactobacillus casei, Lactobacillus reuteri, Lactobacillus acidophilus, Lactobacillus crispatus, Lactobacillus gallinarum, Lactobacillus brevis, Lactobacillus fermentum, Lactobacillus johnsonii and the like, preferably Lactobacillus plantarum, and more preferably Lactobacillus plantarum Viniflora strain (Nova Co.). Examples of the lactic acid bacteria belonging to the genus Oenococcus include Oenococcus oeni, and more preferably Oenococcus oeni PN4 strain, Examples of lactic acid bacteria belonging to the genus Pediococcus include Pediococcus damnosus, Pediococcus pentsaceus, and the like. Examples of the lactic acid bacteria belonging to the genus Lactococcus include Lactococcus lactis, Lactococcus lactis subsp. lactis, Lactococcus garvieae, Lactococcus lactis subsp. cremoris, and Lactococcus lactis subsp, hordniae. Examples of the combination of lactic acid bacteria belonging to the genus Lactobacillus and lactic acid bacteria belonging to the genus Oenococcus include a combination of Lactobacillus plantarum and Oenococcus oeni, and preferably a combination of Lactobacillus plantarum 030701 strain (THT Co.) and CO-INOCULANT BACTERIA (Anchor Co.) (Lactobacillus plantarum and Oenococcus oeni).

The addition amount of lactic acid bacteria based on the coffee cherry pulp and skin extract liquid is, for example, 0.0001 to 1 w/v %, and preferably 0.001 to 0.1 w/v %.

If the lactic acid bacteria are dried, it can be rehydrated by a suitable method.

Fermentation conditions for lactic acid bacteria are not particularly limited as long as fermentation can be carried out, and as necessary, there can be appropriately set conditions suitable for fermentation (for example, type or amount (initial amount) of lactic acid bacteria to be used, amount (concentration) of coffee cherry pulp and skin extract liquid, temperature, pH, oxygen or carbon dioxide concentration, fermentation time, and the like). The fermentation temperature is, for example, 10 to 40° C., and preferably 20 to 30° C., The fermentation time is not particularly limited, but may be appropriately selected depending on the quality of the coffee cherry pulp and skin extract liquid used or the type or amount of lactic acid bacteria. Further, the fermentation step may be completed based roughly on the depletion of the extract liquid. Here, the fermentation time is, for example, 1 to 10 days, and preferably 2 to 8 days.

Further, in the fermentation step using lactic acid bacteria in the present invention, the fermentation process can be carried out using equipment/devices (for example, a constant temperature bath, a tank, a storage house, and the like) which can control automatically and/or manually the above fermentation conditions (for example, type or amount (initial amount) of lactic acid bacteria to be used, amount (concentration) of coffee cherry pulp and skin extract liquid, temperature, pH, oxygen or carbon dioxide concentration, fermentation time, and the like),

### <Step of Yeast Fermentation of Coffee Cherry Pulp and Skin Extract Liquid or Coffee Cherry Pulp and Skin>

The method for producing the extracted and fermented composition of coffee cherry pulp and skin of the present invention may further include a yeast fermentation process. Hereinafter, the step of yeast fermentation of the coffee cherry pulp and skin extract liquid will be described. However, the yeast fermentation process may be performed by yeast fermentation of the pulp and skin of coffee cherry before the pulp and skin extraction process. Those skilled in the art can understand that the extracted and fermented composition of coffee cherry pulp and skin can be produced using each of the processes even in a different order,

According to a preferred embodiment of the present invention, the step of yeast fermentation of the coffee cherry pulp and skin extract liquid is not particularly limited, but includes a method for yeast fermentation by mixing the coffee cherry pulp and skin extract liquid and the yeast and culturing the yeast in the extract liquid.

The step of yeast fermentation of the coffee cherry pulp and skin extract liquid is preferably after the fermentation step using lactic acid bacteria. In such a case, the coffee cherry pulp and skin extract liquid to be subjected to yeast fermentation is a fermented liquid using lactic acid bacteria of the coffee cherry pulp and skin extract liquid.

The yeast used for fermentation is not particularly limited as long as it does not disturb the effects of the present invention, but Saccharomyces yeast, Kluyveromyces yeast, and Torulaspora yeast may be used alone or in combination. The preferred yeast is Saccharomyces yeast. Examples of the yeast belonging to the genus Saccharomyces include Saccharomyces cerevisiae and the like, and more preferably Saccharomyces cerevisiae VIN13 strain. Examples of the yeast of the genus Kluyveromyces include Kluyveromyces thermotolerans and the like. Examples of the yeast of the genus Torulaspora include Torulaspora delbrueckii and the like.

The addition amount of yeast based on the coffee cherry pulp and skin extract liquid is, for example, 0,0001 to 1 w/v %, preferably 0.001 to 0.1 w/v %.

If the yeast is dried, it can be rehydrated by a suitable method.

Fermentation conditions for yeast are not particularly limited as long as fermentation can be carried out, and as necessary, there can be appropriately set conditions suitable for fermentation (for example, type or amount (initial amount) of yeast to be used, amount (concentration) of coffee cherry pulp and skin extract liquid, presence or absence of fermentation of the extract liquid, temperature, pH, oxygen or carbon dioxide concentration, fermentation time, and the like). The fermentation temperature is, for example, 5 to 35° C., and preferably 15 to 25° C. The fermentation time is not particularly limited, but may be appropriately selected depending on the quality (for example, presence or absence of fermentation) of the coffee cherry pulp and skin extract liquid used or the type or amount of yeast. Further, the fermentation step may be completed based roughly on the depletion of the extract liquid. Here, the fermentation time is, for example, 1 to 10 days, and preferably 2 to 8 days.

Further, in the yeast fermentation step in the present invention, the fermentation process can be carried out using equipment/devices (for example, a constant temperature bath, a tank, a storage house, and the like) which can control automatically and/or manually the above fermentation conditions (for example, type or amount (initial amount) of yeast to be used, amount (concentration) of coffee cherry pulp and skin extract liquid, temperature, pH, oxygen or carbon dioxide concentration, fermentation time, and the like).

<Step of Treating Coffee Cherry Pulp and Skin Extract Liquid with a Filter Aid>

The method for producing an extracted and fermented composition of coffee cherry pulp and skin of the present invention may further include a treatment with a filter aid. Hereinafter, the step of treating the coffee cherry pulp and skin extract liquid with a filter aid will be described. However, the treatment with filter aid may be performed by treating the coffee cherry pulp and skin extract liquid with a filter aid after the fermentation process. Those skilled in the art can understand that the extracted and fermented composition of coffee cherry pulp and skin can be produced using each of the processes even in a different order.

The step of treating the coffee cherry pulp and skin extract liquid with a filter aid is not particularly limited, but is, for example, a step of mixing the coffee cherry pulp and skin extract liquid and a filter aid by stirring or the like. The step is advantageous in reducing ingredients such as polyphenols and the like in the coffee cherry pulp and skin extract liquid. Furthermore, it is advantageous to increase the content of J-damascenone in the extracted and fermented composition of coffee cherry pulp and skin obtained by fermentation using lactic acid bacteria and/or yeast, by reducing the ingredients such as polyphenols and the like.

The step of treating the coffee cherry pulp and skin extract liquid with a filter aid is performed preferably before the fermentation step from the viewpoint of increasing the content of β-damascenone in the extracted and fermented composition of coffee cherry pulp and skin.

The filter aid of the present invention can be appropriately selected for the intended purpose by those skilled in the art. The filter aid is not particularly limited as long as it does not disturb the effects of the present invention, but those capable of reducing ingredients such as polyphenois and the like are preferable, and specific examples thereof include polyvinylpolypyrrolidone (PVPP), diatomaceous earth and the like. From the viewpoint of removal of polyphenols, PVPP is more preferable.

The treatment conditions for using the filter aid can be appropriately set for the purpose by those skilled in the art. Such conditions are not particularly limited, but conditions that can reduce ingredients such as polyphenols and the like in the coffee cherry pulp and skin extract liquid are preferable, and there can be appropriately set conditions suitable for process (for example, type or amount of filter aid to be used, amount (concentration) of the coffee cherry pulp and skin extract liquid, temperature, pH, process time, and the like). The amount of the filter aid is, for example, 0.1 to 10 w/v %, and preferably 0.5 to 5 w/v %. Examples of the processing time include 10 minutes to 5 hours, and preferably 30 minutes to 2 hours.

It is preferable to remove the filter aid together with the ingredients adsorbed on the filter aid after the treatment with the filter aid. Examples of such a removing method include centrifugation, filtration, and the like.

### <Step of Concentrating Coffee Cherry Pulp and Skin Extract Liquid>

The method for producing an extracted and fermented composition of coffee cherry pulp and skin of the present invention may further include a concentration process. Hereinafter, the step of concentrating the coffee cherry pulp and skin extract liquid will be described. However, the coffee cherry pulp and skin extract liquid may be subjected to the concentration process after fermentation. Those skilled in the art can understand that the extracted and fermented composition of coffee cherry pulp and skin can be produced using each of the processes even in a different order.

The step of concentrating the coffee cherry pulp and skin extract liquid is not particularly limited, and may include well-known methods such as membrane concentration (for example, ultrafiltration, reverse osmosis method), evaporation concentration (for example, concentration under reduced pressure, vacuum concentration), and centrifugal concentration, cooling concentration and the like, and preferably evaporation concentration. The concentration can be performed for a liquid amount (mass) to be reduced to, for example, ½ to 1/10, and preferably ¼ to ⅙.

The step of concentrating the coffee cherry pulp and skin extract liquid is preferably performed before the step of fermenting the coffee cherry pulp and skin extract liquid using lactic acid bacteria. The concentration step may be performed either before or after the step of treating the coffee cherry pulp and skin extract liquid with a filter aid, but from the viewpoint of the load of concentration step, is preferably performed after the step of treatment with a filter aid.

According to a preferred embodiment of the present invention, in the method for producing an extracted and fermented composition of coffee cherry pulp and skin, each step is preferably carried out in the following sequence.

(1) Step of producing a coffee cherry pulp and skin extract liquid
(2) As desired, step of treating the coffee cherry pulp and skin extract liquid with a filter aid.
(3) As desired, step of concentrating the coffee cherry pulp and skin extract liquid
(4) Step of fermenting the coffee cherry pulp and skin extract liquid using lactic acid bacteria
(5) As desired, step of yeast fermenting the fermented liquid using lactic acid bacteria of the coffee cherry pulp and skin extract liquid.

According to another preferred embodiment of the present invention, in the method for producing an extracted and fermented composition of coffee cherry pulp and skin, each step is carried out in the following order.

(1) Step of fermenting pulp and skin of coffee cherries using lactic acid bacteria
(2) Step of producing an extract liquid of the fermented coffee cherry pulp and skin
(3) As desired, step of treating the extract liquid of the fermented coffee cherry pulp and skin with a filter aid
(4) As desired, step of concentrating the extract liquid of the fermented coffee cherry pulp and skin
(5) As desired, step of yeast fermenting the extract liquid of the fermented coffee cherry pulp and skin.

According to another preferred embodiment of the present invention, in the method for producing an extracted and fermented composition of coffee cherry pulp and skin, each step is carried out in the following order.

(1) Step of fermenting pulp and skin of coffee cherries using lactic acid bacteria
(2) As desired, step of yeast fermenting the fermented coffee cherry pulp and skin using lactic acid bacteria
(3) Step of producing an extract liquid of the fermented coffee cherry pulp and skin
(4) As desired, step of treating the extract liquid of the fermented coffee cherry pulp and skin with a filter aid
(5) As desired, step of concentrating the extract liquid of the fermented coffee cherry pulp and skin

According to another preferred embodiment of the present invention, in the method for producing an extracted and fermented composition of coffee cherry pulp and skin, each step is carried out in the following order.

(1) Step of fermenting pulp and skin of coffee cherries using yeast
(2) Step of fermenting the fermented coffee cherry pulp and skin using lactic acid bacteria
(3) Step of producing an extract liquid of the fermented coffee cherry pulp and skin
(4) As desired, step of treating the extract liquid of the fermented coffee cherry pulp and skin with a filter aid
(5) As desired, step of concentrating the extract liquid of the fermented coffee cherry pulp and skin

The extracted and fermented composition of coffee cherry pulp and skin obtained by the production method of the present invention is also included in the present invention,

### <Food and Drink Containing Extracted and Fermented Composition of Coffee Cherry Pulp and Skin>

The food and drink of the present invention may be prepared by using the composition of the present invention itself as a food and drink, or further blending various proteins, sugars, fats, micronutrients, vitamins, and the like, or adding the composition to a general food and drink. Since the composition of the present invention has a sweet fragrance like honey, it is advantageous in imparting such a fragrance to a food and drink.

The content of the composition of the present invention in the food and drink is not particularly limited, but can be, for example, 0.001 to 100 mass % in the food and drink, and preferably 0.1 to 10 mass %.

Examples of such food and drink include beverages such as alcoholic beverages and non-alcoholic beverages; carbohydrate-containing food and drink such as rice, noodles, breads and pasta; various confectioneries such as Western confectioneries such as cookies and cakes, Japanese confectioneries such as manju and yokan, candies, gums, chilled sweets such as yogurt and pudding, frozen desserts, processed products using eggs, processed products (including delicacies) of seafood (squid, octopus, shellfish, eel, and the like) and meat (including guts such as liver). Specific examples of the above-mentioned alcoholic beverages include whiskey, bourbon, spirits, liqueur, wine, fruit wine, sake, Chinese liquor, shochu, beer, non-alcoholic beer with an alcohol content of 1% or less, non-alcoholic shochu and soda, and non-alcoholic wine, low-malt beer, other miscellaneous liquor, shochu and soda, and the like. The non-alcoholic beverages mentioned above include fruit juice-containing beverages, vegetable juice-containing beverages, fruit juice and vegetable juice-containing beverages, carbonated beverages such as cider, lemonade beverages, cola beverages and the like, soft drinks, milk, soy milk, milk beverages, drink-type yogurt, drink-type jelly, coffee, cocoa, tea beverages, nutritional drinks, sports beverages, mineral water, beer-taste beverages, alcohol-taste beverages, and the like. Preferred aspects of the food and drink of the present invention are beverages such as alcoholic beverages, non-alcoholic beverages, and the like, and more preferably wine, shochu and soda and carbonated beverages.

According to one embodiment of the present invention, the beverage of the present invention may be a beverage other than coffee beverages (beverages made using green coffee beans as raw material),

### <Method for Increasing β-Damascenone Content in Extracted and Fermented Composition of Coffee Cherry Pulp and Skin>

The present invention includes subjecting pulp and skin of coffee cherries to an extraction process and a fermentation process using lactic acid bacteria, and preferably includes a step of fermenting a coffee cherry pulp and skin extract liquid using lactic acid bacteria, which increases the β-damascenone content. Therefore, according to another aspect of the present invention, the present invention is a method for increasing the β-damascenone content in the extracted and fermented composition of coffee cherry pulp and skin, and the method includes subjecting pulp and skin of coffee cherries to an extraction process and a fermentation process using lactic acid bacteria. According to another preferred aspect of the present invention, there is provided a method for increasing the β-damascenone content in the extracted and fermented composition of coffee cherry pulp and skin, and the method includes a step of fermenting a coffee cherry pulp and skin extract liquid using lactic acid bacteria.

### EXAMPLES

Hereinafter, the present invention will be described in more detail with reference to examples, but the present invention is not limited to these examples. In the examples, “%” means “mass %” unless otherwise specified. Further, the unit and the measuring method of the present invention are in accordance with JIS unless otherwise specified.

### Method for Measuring β-Damascenone Content

In order to quantify the amount of β-damascenone in the liquid, the measurement was performed by gas chromatography-mass spectrometry (GC-MS) after pretreatment.

### (1) Preparation of Calibration Curve

The measurement was performed by the internal standard method, and 4-nonanol was used as an internal standard substance. An internal standard substance solution was prepared using a known amount of 4-nonanol and ethanol (50 v/v %). R-Damascenone was used as a reference substance. A reference substance solution was prepared using a known amount of β-damascenone and ethanol (50 v/v %). The reference substance solution and the internal standard substance solution was diluted stepwise with a base sample and used as follows to obtain samples for preparing the calibration curve. Here, there was used the base sample, which was prepared by weighing about 3 g of tartaric acid, dissolving the tartaric acid in ethanol (120 mL) and distilled water (880 mL), and adjusting the solution to pH 3.2.

(i) The reference substance solution (5.0 mL) diluted stepwise with the base sample was separated into a glass test tube, and the internal standard substance solution (50 μL) was added to the tube to prepare a sample for the calibration curve.
(ii) Dichloromethane (1.5 mL) was added to the sample prepared in (i) for the calibration curve, and the mixture was put in a vortex mixer for 2 minutes.
(iii) The sample of (ii) was centrifuged at 3000 rpm, 4° C. for 5 minutes.
(iv) The lower layer (dichloromethane layer) of the sample of (iii) was separated into an Eppendorf tube (2.0 mL volume) using a Pasteur pipette.
(v) The sample of (iv) was dehydrated with anhydrous sodium sulfate. The dehydration was performed twice.
(vi) The sample after completion of dehydration was placed in a vial for GC-MS measurement and subjected to GC-MS measurement.
(vii) A calibration curve was prepared based on the obtained GC-MS measurement results.

### (2) Sample Pretreatment

(i) The sample (the above liquid, 5.0 mL) was separated into a glass test tube, and the internal standard substance solution (50 μL) was added to the tube.
(ii) Dichloromethane (1.5 mL) was added to the sample prepared in (i), and the mixture was put in a vortex mixer for 2 minutes.
(iii) The sample of (ii) was centrifuged at 3000 rpm, 4° C. for 5 minutes.
(iv) The lower layer (dichloromethane layer) of the sample of (iii) was separated into an Eppendorf tube (2.0 mL volume) using a Pasteur pipette.
(v) The sample of (iv) was dehydrated with anhydrous sodium sulfate. The dehydration was performed twice.
(vi) The sample after completion of dehydration was placed in a vial for GC-MS measurement and subjected to GC-MS measurement.

### (3) The Measurement of GC-MS was Carried Out Using the Following Apparatus Under the Following Conditions.

### <Apparatus>

7890B (manufactured by Agilent Technologies, Inc.)

JMS-Q1500 (manufactured by JEOL Ltd.)

### <Column>

BP-20 (manufactured by SGE Analytical Science Pty. Ltd)

### <GC Measurement Conditions>

Oven temperature

Initial: 60° C., hold 3 minutes

Temperature rise: 3° C./min, 170° C., hold 3 minutes,

10° C./min, 245° C., hold 10 minutes

Injection port setting

Injection volume: 2 μL

Mode: Splitless 10:1

Temperature: 25° C.

Pressure: 162.35 kPa

Total flow rate: 1 mL/min
Septum purge flow rate: 3 mL/min
Aux temperature: 240° C.

### <MS Measurement Conditions (SIM/Scan)>

SIM target ion: 190 (β-Damascenone)

Threshold: 10

MS zone temperature

Ion source temperature; 230° C.

Quadrupole temperature: 100° C.

### Test Example 1-1 (Examples 1 and 2, Comparative Examples 1 and 2): Examination of Effects of Lactic Acid Bacteria and Yeast on Fermentation of Coffee Cherry Pulp and Skin Extract Liquid

(1) Water (2250 mL) was added to skin and pulp of coffee cherries (1500 g) (Coffea arabica, produced in Okinawa) after removing coffee beans (coffee seeds), and the mixture was allowed to stand at 60° C. for 1 hour. Then, the juice was squeezed using a juicer to separate the extract liquid from the fruit to obtain a coffee cherry pulp and skin extract liquid. Only outer skin and pulp was used for the skin and pulp.

(2) A suspension liquid of lactic acid bacteria (10 w/v %) in distilled water was added to the liquid obtained in (1) so that the amount of lactic acid bacteria became 100 ppm (=0.01 w/v %), and the mixture was incubated at 25° C. for 5 days to obtain a fermented liquid using lactic acid bacteria. In Example 1, Oenococcus oeni PN4 strain (THT Co.) was used as the lactic acid bacteria. In Example 2, Lactobacillus plantarum Viniflora strain (NOVA Co.) was used as the lactic acid bacteria. In Comparative Examples 1 and 2, lactic acid bacteria were not added.

(3) After that, yeast liquid (10 w/v %) was added to the liquid obtained in (2) so that the amount of yeast became 100 ppm (=0.01 w/v %), and the mixture was incubated at 20° C. for 5 days to obtain a fermented liquid using lactic acid bacteria and yeast. In Examples 1 and 2 and Comparative Example 2, Saccharomyces cerevisiae VIN13 strain (Anchor Co.) was used as the yeast. In Comparative Example 1, yeast was not added.

(4) The amount of β-damascenone in the liquid obtained in (3) was quantified,

The test results of Test Example 1-1 (Examples 1 and 2, Comparative Examples 1 and 2) are shown in Table 1.

From the results in Table 1, the β-damascenone content was found to increase by fermentation using yeast and lactic acid bacteria. The β-damascenone content was also found to further increase by using Lactobacillus lactic acid bacteria as the lactic acid bacteria.

TABLE 1

| | | | | | | Comparative | Comparative |
|---|---|---|---|---|---|---|---|
| | Example 1 | Example 2 | Example 3 | Example 4 | Example 5 | Example 1 | Example 2 |
| Extraction | 60 | 60 | 60 | 60 | 60 | 60 | 60 |
| temperature | | | | | | | |
| Extraction | Extraction for 1 h −> Squeeze | | | | | | |
| method | | | | | | | |
| Concentration | none | | | | | | |
| treatment with | — | — | PVPP 1% | — | PVPP 1% | — | — |
| filter aid | | | 1 h | | 1 h | | |
| Fermentation | Liquid fermentation | | | | | | |
| method | | | | | | | |
| Lactic acid | O. oeni | L. plantarum | L. plantarum | L. plantarum | L. plantarum | — | — |
| bacteria | PN4 | Viniflora | Viniflora | Viniflora | Viniflora | | |
| Amount of | 100 ppm | 100 ppm | 100 ppm | 100 ppm | 100 ppm | | |
| lactic acid | | | | | | | |
| bacteria | | | | | | | |
| Yeast | S. cerevisiae | S. cerevisiae | S. cerevisiae | — | — | — | S. cerevisiae |
| | VIN13 | VIN13 | VIN13 | | | | VIN13 |
| β-Damascenone | 12.55 | 18.41 | 22.85 | 12.99 | 18.52 | 0.19 | 8.64 |
| (ppb) | | | | | | | |

### Test Example 1-2 (Example 3): Examination of Effect of PVPP on Fermentation of Coffee Cherry Pulp and Skin Extract Liquid

(1) In the same process as in (1) of Test Example 1-1, a coffee cherry pulp and skin extract liquid was obtained.

(2) To the coffee cherry pulp and skin extract liquid obtained in (1), polyvinylpolypyrrolidone (PVPP, 1 w/v %) (PVPP granules, manufactured by Hashima Karyu Kogyo Co., Ltd.) was added, and the mixture was stirred using a stirrer for 1 hour. Then, the mixture was centrifuged using a centrifuge at 5000 rpm for 10 minutes, and only the supernatant was transferred to another container.

(3) The supernatant obtained in (2) was subjected to the same process as in (2) of Test Example 1-1 to obtain a fermented liquid using lactic acid bacteria. In Example 3, Lactobacillus plantarum Viniflora strain (Nova Co.) was used as the lactic acid bacteria.

(4) The liquid obtained in (3) was subjected to the same process as in (3) of Test Example 1-1 to obtain a fermented liquid using lactic acid bacteria and yeast. In Example 3, Saccharomyces cerevisiae VIN13 strain was used as the yeast.

(5) The amount of β-damascenone was quantified in the fermented liquid using lactic acid bacteria and yeast obtained in (4).

The test results of Test Example 1-2 (Example 3) are shown in Table 1.

From the results in Table 1, the 3-damascenone content was found to increase by using PVPP.

### Test Example 1-3 (Examples 4 and 5): Examination of Effect of Yeast on Fermentation of Coffee Cherry Pulp and Skin Extract Liquid

(1) In the same process as in (1) of Test Example 1-1, a coffee cherry pulp and skin extract liquid was obtained.

(2) The same process as in (2) of Test Example 1-2 was not performed for Example 4 and was performed for Example 5.

(3) The same process as in (2) of Test Example 1-1 was performed to the coffee cherry pulp and skin extract liquid obtained in (1) for Example 4, and to the supernatant obtained in (2) for Example 5, to obtain a fermented liquid using lactic acid bacteria for each Example. In Examples 4 and 5, Lactobacillus plantarum Viniflora strain was used as the lactic acid bacteria.

(4) The amount of β-damascenone was quantified in the fermented liquid using lactic acid bacteria obtained in (3).

The test results of Test Examples 1-3 (Examples 4 and 5) are shown in Table 1.

From the results in Table 1, the β-damascenone content was found to increase even by lactic acid bacteria alone.

### Test Example 2 (Examples 6 and 7): Examination of Effect of Concentration on Fermentation of Coffee Cherry Pulp and Skin Extract Liquid

(1) The same process as in (1) of Test Example 1-1 was performed to obtain a coffee cherry pulp and skin extract liquid.

(2) The same process as in (2) of Test Example 1-2 was performed.

(3) The supernatant obtained in (2) from the coffee cherry pulp and skin extract liquid after the PVPP process was concentrated for Example 7. Specifically, the supernatant (500 g) of the coffee cherry pulp and skin extract liquid after the PVPP process was concentrated using an evaporator at 70° C. under reduced pressure until the concentrated side became 100 g. No concentration was performed for Example 6.

(4) The same process as in (2) of Test Example 1-1 was performed to the supernatant obtained in (2) for Example 6 and to the liquid obtained in (3) for Example 7, to obtain a fermented liquid using lactic acid bacteria for each Example. In Examples 6 and 7, Lactobacillus plantarum 030701 strain (THT Co.) and CO-INOCULANT BACTERIA (Oenococcus oeni/Lactobacillus plantarum blend) (Anchor Co.) were used as the lactic acid bacteria.

(5) The liquid obtained in (4) was subjected to the same process as in (3) of Test Example 1-1 to obtain a fermented liquid using lactic acid bacteria and yeast. Saccharomyces cerevisiae VIN13 strain was used as the yeast for Examples 6 and 7,

(6) The amount of β-damascenone was quantified in the fermented liquid using lactic acid bacteria and yeast obtained in (5).

The test results of Test Example 2 (Examples 6 and 7) are shown in Table 2.

TABLE 2

| | Example 6 | Example 7 |
|---|---|---|
| Extraction | 60 | 60 |
| temperature | | |
| Extraction method | Extraction for 1 h -> Squeeze | |
| Concentration | none | present |
| treatment with fitter | PVPP 1% 1 h | PVPP 1% 1 h |
| aid | | |
| Fermentation method | Liquid fermentation | |
| Lactic acid bacteria | L. plantarum | L. plantarum |
| | 030701 | 030701 |
| | + | + |
| | CO-INOCULANT | CO-INOCULANT |
| | BACTERIA | BACTERIA |
| Amount of lactic acid | 100 ppm | 100 ppm |
| bacteria | | |
| Yeast | S. cerevisiae | S. cerevisiae |
| | VIN13 | VIN13 |
| β-Damascenone | 16.24 | 15.75 |
| (ppb) | | |

From the results in Table 2, no difference due to presence or absence of concentration was observed in the amount of β-damascenone.

1. An extracted and fermented composition of coffee cherry pulp and skin, comprising β-damascenone at 9.6 ppb or more.

2. The extracted and fermented composition of coffee cherry pulp and skin according to claim 1, wherein the composition contains 50 times or more of β-damascenone as compared to an unfermented coffee cherry pulp and skin extract liquid.

3. A method for producing an extracted and fermented composition of coffee cherry pulp and skin, comprising subjecting pulp and skin of coffee cherry to an extraction process and a fermentation process using lactic acid bacteria.

4. The method according to claim 3, wherein the method comprises a step of fermenting a coffee cherry pulp and skin extract liquid using lactic acid bacteria.

5. The method according to claim 3, wherein the lactic acid bacteria are at least one selected from the group consisting of the genus Lactobacillus, the genus Oenococcus, the genus Pediococcus, and the genus Lactococcus.

6. The method according to claim 3, further comprising a yeast fermentation process.

7. The method according to claim 3, wherein the method comprises a step of fermenting using yeast after the step of fermenting a coffee cherry pulp and skin extract liquid using lactic acid bacteria.

8. The method according to claim 3, further comprising a treatment with a filter aid.

9. The method according to claim 3, further comprising a step of treating a coffee cherry pulp and skin extract liquid with a filter aid before the step of fermenting the coffee cherry pulp and skin extract liquid using lactic acid bacteria.

10. The method according to claim 3, further comprising a concentration process.

11. The method according to claim 3, further comprising a step of concentrating a coffee cherry pulp and skin extract liquid before the step of fermenting the coffee cherry pulp and skin extract liquid using lactic acid bacteria.

12. An extracted and fermented composition of coffee cherry pulp and skin, obtained by the method according to claim 3.

13. A beverage comprising the extracted and fermented composition of coffee cherry pulp and skin according to claim 1.

14. The beverage according to claim 13, wherein the beverage is a packaged beverage.

15. A method for increasing a β-damascenone content in an extracted and fermented composition of coffee cherry pulp and skin, comprising subjecting coffee cherry pulp and skin to an extraction process and a fermentation process using lactic acid bacteria.

16. The method according to claim 15, wherein the method comprises a step of fermenting a coffee cherry pulp and skin extract liquid using lactic acid bacteria.
